(19) United States
(12) Patent Application Publication
(10) Pub. No.: US 2019/0374774 A1
(43) Pub. Date: Dec. 12, 2019

(54) IMPLANTABLE EXTRAOCULAR MUSCLE NEUROMUSCULAR STIMULATOR AND PARAMETER SETTING METHOD THEREFOR

(71) Applicant: LEJIN WANG, Beijing (CN)

(72) Inventors: LEJIN WANG, BEIJING (CN); ZEQUN MIAO, BEIJING (CN); YAO LU, BEIJING (CN); XIAOLI MENG, BEIJING (CN); LILI GUO, BEIJING (CN); JIAMEI DONG, BEIJING (BE); LUOJIA LI, BEIJING (CN)

(73) Assignee: Lejin Wang

(21) Appl. No.: 16/546,309

(22) Filed: Aug. 21, 2019

Related U.S. Application Data
(63) Continuation of application No. PCT/CN2017/081942, filed on Apr. 26, 2017.

(30) Foreign Application Priority Data
Feb. 21, 2017 (CN) 201710092691.6

(51) Int. Cl. A61N 1/36; A61N 1/375

(52) U.S. Cl. CPC A61N 1/36046; A61N 1/3752; A61N 1/3606; A61N 1/36139; A61N 1/36125

(57) ABSTRACT

The present application discloses an implantable extraocular-muscle neuromuscular stimulator and its parameter setting method, the implantable extraocular-muscle neuromuscular stimulator comprising an implantable pulse generator, an extracorporal remote controller and an electrode, wherein the implantable pulse generator is provided in an created subcutaneous pouch, the implantable pulse generator is connected to an extraocular muscle by the electrode, and the implantable pulse generator is for generating an pulse stimulating signal; and the extracorporal remote controller is connected to the implantable pulse generator by a wireless signal, and is for setting the pulse stimulating signal of the implantable pulse generator. The implantable extraocular-muscle neuromuscular stimulator and its parameter setting method provided by the present application are applied to the treatment of congenital idiopathic nystagmus.

### CROSS-REFERENCE TO RELATED APPLICATIONS

This application is a continuation of International Patent Application No. PCT/CN2018/152954 with a filing date of Apr. 26, 2017, designating the United States, now pending, and claims priority to Chinese Patent Application No. 201710092691.5 with a filing date of Feb. 21, 2017. The content of the aforementioned applications, including any intervening amendments thereto, are incorporated herein by reference.

### TECHNICAL FIELD

The present application relates to the field of miniature medical equipment, and particularly relates to an implantable extraocular-muscle neuromuscular stimulator and its parameter setting method.

### BACKGROUND ART

Nystagmus is involuntary, rhythmic and reciprocal eyeball swinging or jumping, and it has complicated clinical manifestation, and is frequently accompanied with visual impairment of both eyes. According to the primary diseases of nystagmus, nystagmus is classified as types such as ocular nystagmus, vetibular nystagmus, central nystagmus and cryptogenic nystagmus.

Among them, ocular nystagmus is clinically closely relevant to ophthalmology, and is mainly congenital nystagmus (CN), which frequently emerges at birth or within 4 months after birth. According to statistics, its prevalence rate among children is 1/1000-1/1500, and globally there are approximately millions of patients. According to the etiologies, it is classified as: Sensory Defect Nystagmus (SDN) and Congenital Idiopathic Nystagmus (CIN).

CIN is congenital defective outgoing-mechanism and normal passing mechanism, and the abnormality of non-closing eyes is frequently expressed as impulsive nystagmus. The main harm of CIN is resulting in amblyopia. According to statistics, among the patients, the cases of illness of amblyopia account for 86.7%, wherein moderate and severe amblyopia account for 20.2%, and moderate amblyopia accounts for 52.3%. Accordingly, it can be seen that, CIN affects vision widespreadly.

Because the particular pathogenesis of CIN has not been clear, and there is no clinically effective approach, the disease is always one of the difficult and key diseases in ophthalmology.

Implantable medical-treatment instruments are diverse, such as cardiac pacemaker, defibrillator and implantable muscle stimulator. The present application relates to an implantable extraocular-muscle neuromuscular stimulator and its parameter setting method for treating congenital idiopathic nystagmus.

### SUMMARY OF THE DISCLOSURE

Regarding the above problems, the present application proposes an implantable extraocular-muscle neuromuscular stimulator and its parameter setting method, for treating congenital idiopathic nystagmus. The stimulator, by detecting the contraction state of the extraocular muscle and the stimulating signal, supplies the extraocular muscle the corresponding stimulation and contraction, to balance the nerve stimulation and counteract the contraction, to enable the eyeballs to maintain fixed staring and not to have nystagmus.

The technical solution of the present application:

In order to solve the above technical problem, the present application provides an implantable extraocular-muscle neuromuscular stimulator, comprising an implantable pulse generator, an extracorporal remote controller and an electrode, wherein the implantable pulse generator is provided in an created subcutaneous pouch, the implantable pulse generator is connected to an extraocular muscle by the electrode, and the implantable pulse generator is for generating an pulse stimulating signal; the electrode comprises a lead and a contact-pin connector and a contact that are provided at two ends of the lead, wherein the contact-pin connector is connected to the implantable pulse generator, the lead is provided in a created subcutaneous tunnel, and the contact is connected to the extraocular muscle; and the extracorporal remote controller is connected to the implantable pulse generator by a wireless signal, and is for setting the pulse stimulating signal of the implantable pulse generator; wherein the implantable pulse generator is for sensing a contraction state of an eye muscle by using the electrode, and according to the contraction state of the eye muscle generating an eye-muscle stimulating signal, and sending the eye-muscle stimulating signal to the extracorporal remote controller; and the implantable pulse generator is further for receiving an eye-muscle stimulating signal of an opposite direction that is emitted by the extracorporal remote controller, and outputting the eye-muscle stimulating signal by using the electrode to another eye muscle. Optionally, the implantable pulse generator comprises a receiver, a transmitter, a wireless energy transfer module, a power management module, a digital processing module and an electrical-stimulation driver, and the wireless energy transfer module transmits energy to the power management module, the receiver and the transmitter; and the implantable pulse generator wirelessly receives the signal by using the receiver, and transmits an output to the digital processing module, the signal is transmitted to the electrical-stimulation driver via a current generating circuit and a waveform time-sequence circuit, and the electrical-stimulation driver is connected to the electrode.

Optionally, the extracorporal remote controller comprises a receiver, a transmitter, a frequency synthesizer, a digital processing module, a data analysis module, a data storage module, a display and a processor, the processor is connected to the data analysis module, the data storage module, a controlling program and the display, and the frequency synthesizer is connected to the digital processing module, the receiver and the transmitter; the extracorporal remote controller wirelessly receives the signal by using the receiver, and transmits the signal to the digital processing module, and the signal is transmitted to the processor via a digital interface circuit; and the processor transmits the signal to the data analysis module, and stores an analysis result to the data storage module, and the processor transmits the analysis result to the implantable pulse generator via the digital interface circuit, the digital processing module and the transmitter.

Optionally, the contact on the electrode is a plurality of contacts, and when the contacts are connected to the extraocular muscle, at least one of the contacts is connected to the extraocular muscle; and the contact is connected to the extraocular muscle by binding or insertion.

Optionally, the setting of the pulse stimulating signal comprises: pulse amplitude setting, pulse width setting and pulse frequency setting.

Optionally, the pulse amplitude setting comprises a voltage mode and a current mode, wherein when a pulse amplitude is set in the voltage mode, a voltage range is 0-10V, and an adjustment step length is 1V or 0.1V; and when a pulse amplitude is set in the current mode, a current range is 0-25 mA, and an adjustment step length is 1 mA or 0.1 mA.

Optionally, a range of the pulse width setting is 30-450 ms, and an adjustment step length is 10 ms.

Optionally, a range of the pulse frequency setting is 1-500 Hz.

The present application further discloses a method for setting a parameter of the implantable extraocular-muscle neuromuscular stimulator, wherein the setting the pulse stimulating signal of the implantable pulse generator by using the extracorporal remote controller comprises: pulse amplitude setting, pulse width setting and pulse frequency setting.

Optionally, the pulse amplitude setting comprises a voltage mode and a current mode, wherein when a pulse amplitude is set in the voltage mode, a voltage range is 0-10V, and an adjustment step length is 1V or 0.1V; and when a pulse amplitude is set in the current mode, a current range is 0-25 mA, and an adjustment step length is 1 mA or 0.1 mA; a range of the pulse width setting is 30-450 ms, and an adjustment step length is 10 ms; and a range of the pulse frequency setting is 1-500 Hz.

The advantageous effects of the present application:

The implantable extraocular-muscle neuromuscular stimulator and its parameter setting method provided by the present application are applied to the treatment of congenital idiopathic nystagmus. The stimulator, by detecting the contraction state of the extraocular muscle and the stimulating signal, supplies the extraocular muscle the corresponding stimulation and contraction, to balance the nerve stimulation and counteract the contraction, to enable the eyeballs to maintain fixed staring and not to have nystagmus.

### BRIEF DESCRIPTION OF THE DRAWINGS

By the following detailed description made by referring to the drawings, the above and/or other aspects and advantages of the present application will become clearer and easier to understand. The drawings are merely illustrative, and do not limit the present application. In the drawings:

FIG. 1 is a schematic structural diagram of the present application;

FIG. 2 is a module diagram of the implantable pulse generator of the present application;

FIG. 3 is a module diagram of the extracorporal remote controller of the present application;

FIG. 4 is a schematic structural diagram of the electrode of the present application; and

FIG. 5 is a schematic diagram of the application in nystagmus of the present application.

In the drawings, the reference numbers indicate the following components:

1. implantable pulse generator; 2. extracorporal remote controller; 3. electrode; 4. extraocular muscle; 5. contact-pin connector; 6. contact; and 7. lead.

### PARTICULAR EMBODIMENTS

The implantable extraocular-muscle neuromuscular stimulator and its parameter setting method of the present application are described in detail below by referring to the particular embodiments and the drawings.

The embodiments set forth herein are particular embodiments of the present application, are used to explain the concept of the present application, are all explanatory and exemplary, and should not be interpreted as limiting the embodiments of the present application and the scope of the present application. Besides the embodiments set forth herein, a person skilled in the art can employ other apparent technical solutions on the basis of the contents disclosed by the claims and the description of the present application, and those technical solutions include technical solutions that make any apparent substitutions and amendments by using the embodiments set forth herein.

The drawings of the description are schematic diagrams, to auxiliarily explain the concept of the present application, and schematically illustrate the shapes of and the relations between the parts. It should be noted that, in order to facilitate clarity illustrating the structures of the components of the embodiments of the present application, the drawings are not drawn in the same proportion. The same reference numbers indicate the same elements.

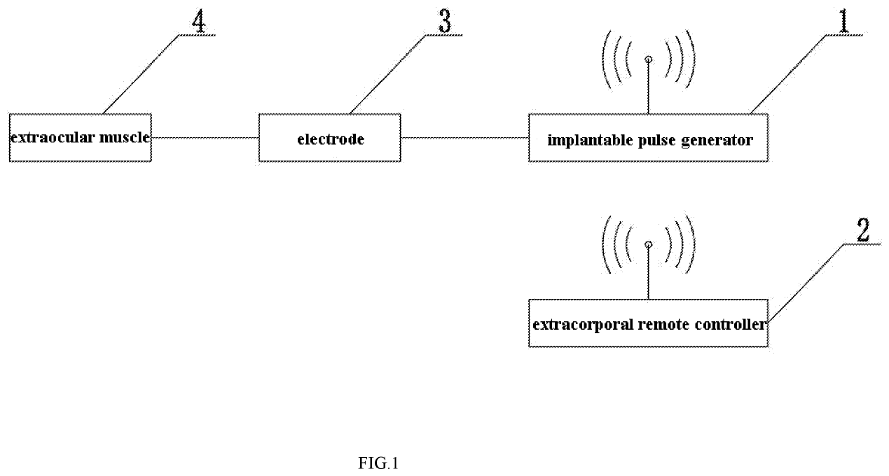

FIG. 1 shows a schematic structural diagram of the implantable extraocular-muscle neuromuscular stimulator. It comprises an implantable pulse generator 1, an extracorporal remote controller 2 and an electrode 3. The implantable pulse generator 1 is provided in a created subcutaneous pouch, the implantable pulse generator 1 is connected to an extraocular muscle 4 by the electrode 3, and the implantable pulse generator 1 is for generating an pulse stimulating signal.

The extracorporal remote controller 2 is connected to the implantable pulse generator 1 by a wireless signal, and is for setting the pulse stimulating signal of the implantable pulse generator 1.

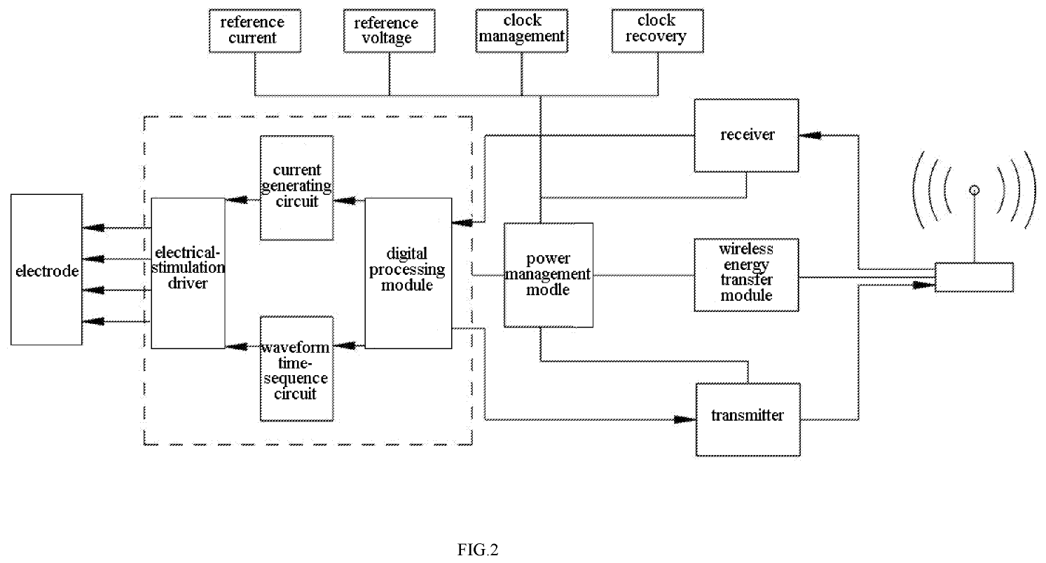

FIG. 2 is a module diagram of the implantable pulse generator of the present application. It comprises a receiver, a transmitter, a wireless energy transfer module, a power management module, a digital processing module and an electrical-stimulation driver, and the wireless energy transfer module transmits energy to the power management module, the receiver and the transmitter; and the implantable pulse generator 1 wirelessly receives the signal by using the receiver, and transmits an output to the digital processing module, the signal is transmitted to the electrical-stimulation driver via a current generating circuit and a waveform time-sequence circuit, and the electrical-stimulation driver is connected to the electrode 3.

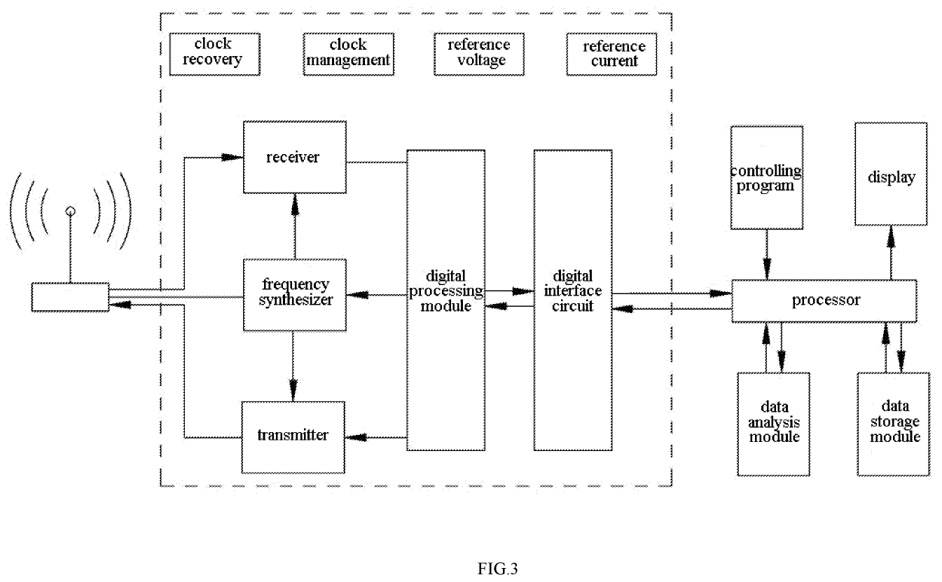

FIG. 3 is a module diagram of the extracorporal remote controller of the present application. The extracorporal remote controller 2 comprises a receiver, a transmitter, a frequency synthesizer, a digital processing module, a data analysis module, a data storage module, a display and a processor, the processor is connected to the data analysis module, the data storage module, a controlling program and the display, and the frequency synthesizer is connected to the digital processing module, the receiver and the transmitter; the extracorporal remote controller 2 wirelessly receives the signal by using the receiver, and transmits the signal to the digital processing module, and the signal is transmitted to the processor via a digital interface circuit; and the processor transmits the signal to the data analysis module, and stores an analysis result to the data storage module, and the processor transmits the analysis result to the implantable pulse generator 1 via the digital interface circuit, the digital processing module and the transmitter.

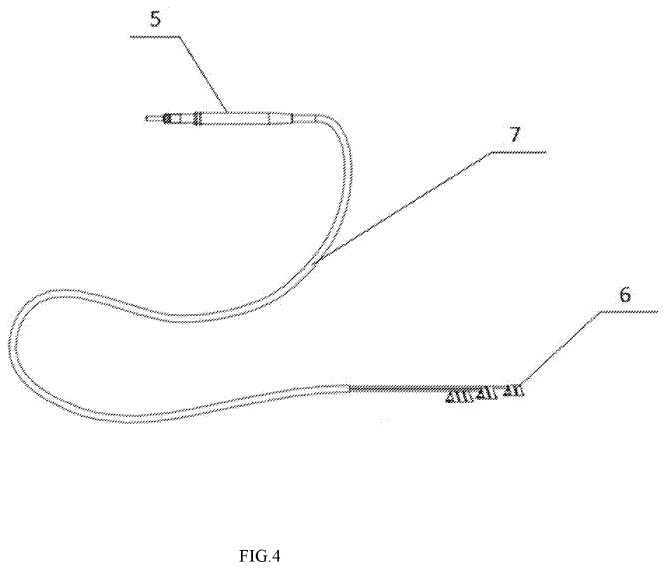

FIG. 4 is a schematic structural diagram of the electrode of the present application. The electrode 3 comprises a lead 7 and a contact-pin connector 5 and a contact 6 that are provided at two ends of the lead 7, wherein the contact-pin connector 5 is connected to a top cover of the implantable pulse generator 1, the lead 7 is provided in a created subcutaneous tunnel, and the contact 6 is connected to the extraocular muscle 4.

The contact 6 on the electrode 3 is a plurality of contacts, and when the contacts 6 are connected to the extraocular muscle 4, at least one of the contacts 6 is connected to the extraocular muscle 4; and the contact 6 is connected to the extraocular muscle 4 by binding or insertion.

The lead 7 is also referred to as an electrode main body, and the length of the lead is generally 20 cm-60 cm.

The present application further provides a method for setting a parameter of the implantable extraocular-muscle neuromuscular stimulator, wherein the setting the pulse stimulating signal by using the extracorporal remote controller 2 comprises: pulse amplitude setting, pulse width setting and pulse frequency setting.

The pulse amplitude setting comprises a voltage mode and a current mode, wherein when a pulse amplitude is set in the voltage mode, a voltage range is 0-10V, and an adjustment step length is 1V or 0.1V; and when a pulse amplitude is set in the current mode, a current range is 0-25 mA, and an adjustment step length is 1 mA or 0.1 mA.

A range of the pulse width setting is 30-450 ms, and an adjustment step length is 10 ms; and a range of the pulse frequency setting is 1-500 Hz.

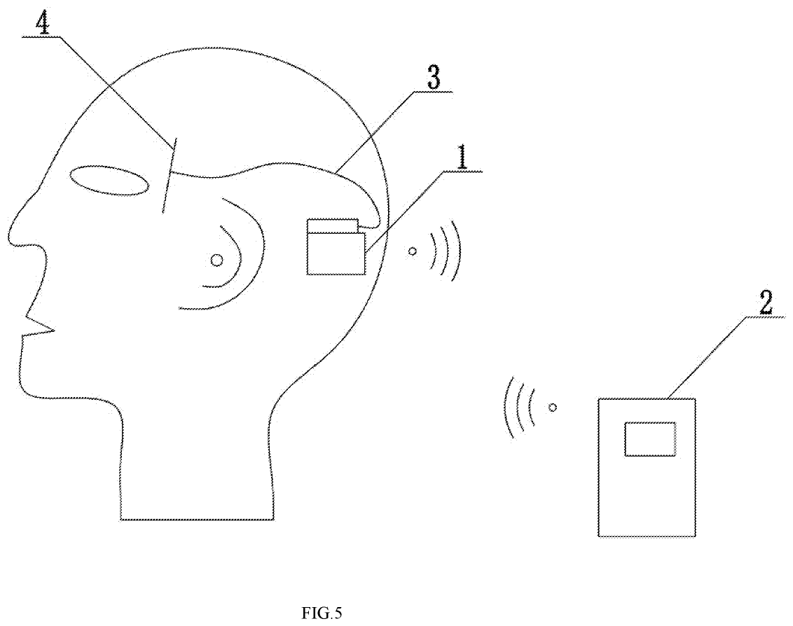

FIG. 5 is a schematic diagram of the application in nystagmus of the present application. The application comprises providing the implantable pulse generator 1 in an subcutaneous pouch behind an ear, binding or inserting the electrode 3 on a peeled extraocular muscle, adjusting the direction of the electrode 3, to avoid the pulling and stress of the electrode 3, and fixing the electrode 3 on the sclera by stitching.

The particular method for treating nystagmus by using the implantable extraocular-muscle neuromuscular stimulator is as follows:

Firstly, when an eyeball of a patient will involuntarily inwardly rotate or outwardly rotate, one of the eye muscles contracts, the implantable pulse generator 1 senses the state of the contraction of that eye muscle by using the electrode 3, analyzes by using the digital processing module on the implantable pulse generator 1 to obtain the degree of the contraction of that eye muscle, and emits an eye-muscle stimulating signal by using the transmitter.

Subsequently, the receiver on the extracorporal remote controller 2 receives the eye-muscle stimulating signal wirelessly, the digital processing module processes the signal, and transmits to the processor, the processor analyzes the eye-muscle stimulating signal by using the data analysis module, and the transmitter on the extracorporal remote controller 2 transmits an opposite stimulating signal.

Subsequently, the implantable pulse generator 1 receives the opposite stimulating signal transmitted by the extracorporal remote controller 2 by using the receiver, and the digital processing module analyzes the signal, and outputs the magnitude and the direction of the stimulating signal that is supplied to the eye muscle.

Subsequently, the electrical-stimulation driver is started up, and the electrode 3 connected to the electrical-stimulation driver supplies the other eye muscle a stimulating signal of the same magnitude and an opposite direction, to cause the other eye muscle to move in the opposite direction and at the same time contract, whereby the forces of the two eye muscles are counteracted, and the eyeball maintains fixed, and does not have nystagmus. Accordingly, the method balances the stimulation on the eye muscles, counteracts the contraction of the eye muscles, and reaches the purpose of treating nystagmus.

When the method is used to treat nystagmus, when an eyeball of a patient will involuntarily inwardly rotate, the medial rectus contracts, the implantable pulse generator 1 senses a contraction signal of the medial rectus, and simultaneously the implantable pulse generator 1 supplies the lateral rectus a stimulating signal of the same magnitude, to cause the lateral rectus to simultaneously contract, whereby the forces of the medial rectus and the lateral rectus are counteracted, and the eyeball maintains fixed, and does not have nystagmus.

In the similar way, when an eyeball of a patient will involuntarily outwardly rotate, the lateral rectus contracts, the implantable pulse generator 1 senses a contraction signal of the lateral rectus, and simultaneously the implantable pulse generator 1 supplies the medial rectus a stimulating signal of the same magnitude, to cause the medial rectus to simultaneously contract, whereby the forces of the lateral rectus and the medial rectus are counteracted, and the eyeball maintains fixed, and does not have nystagmus.

The steps of the surgical method for treating congenital idiopathic nystagmus by using the implantable extraocular-muscle neuromuscular stimulator of the present application are as follows:

S1, peeling to expose the extraocular muscle 4;

S2, binding or inserting the electrode 3 on a peeled extraocular muscle 4;

S3, adjusting the direction of the electrode 3, to avoid the pulling and stress of the electrode 3, and fixing a section of the electrode 3 on the sclera by stitching;

S4, creating a subcutaneous pouch behind an ear for the implantable pulse generator 1;

S5, introducing the electrode 3 via the orbit tunnel and the subcutaneous tunnel to the subcutaneous pouch where the implantable pulse generator 1 is placed;

S6, connecting the implantable pulse generator 1 and the electrode 3, by ensuring that the contact-pin connector 5 on the electrode 3 is inserted into the top cover of the implantable pulse generator 1, and screwing up a fastening screw on the top cover of the implantable pulse generator 1;

S7, performing system test to the implantable pulse generator 1 by using the extracorporal remote controller 2, to confirm that the system is functioning normally;

S8, encasing the implantable pulse generator 1 into the subcutaneous pouch, and Winding the redundant lead around the implantable pulse generator 1;

S9, stitching a stitching hole on the implantable pulse generator 1 and the subcutaneous tissue together, and fixing the implantable pulse generator 1 at the position of the subcutaneous pouch;

S10, performing system test to the implantable pulse generator 1 by using the extracorporal remote controller 2 again, to confirm that the system is functioning normally; and

S11, disinfecting, and stitching the surgical incision.

By the above operation steps, the stimulator, by detecting the contraction state of the extraocular muscle and the stimulating signal, supplies the extraocular muscle the corresponding stimulation and contraction, to balance the nerve stimulation and counteract the contraction, to enable the eyeballs to maintain fixed staring and not to have nystagmus, which effectively treats the disease of congenital idiopathic nystagmus.

The implantable extraocular-muscle neuromuscular stimulator and its parameter setting method provided by the present application are applied to the treatment of congenital idiopathic nystagmus. The stimulator, by detecting the contraction state of the extraocular muscle and the stimulating signal, supplies the extraocular muscle the corresponding stimulation and contraction, to balance the nerve stimulation and counteract the contraction, to enable the eyeballs to maintain fixed staring and not to have nystagmus.

The present application is not limited to the above embodiments. A person skilled in the art can obtain other technical solutions of various forms by the motivation of the present application. However, regardless of any variation in their shapes or structures, any technical solutions that are the same as or similar to the present application fall within the protection scope of the present application.

We claim:

1. An implantable extraocular-muscle neuromuscular stimulator, comprising an implantable pulse generator (1), an extracorporal remote controller (2) and an electrode (3), wherein
the implantable pulse generator (1) is provided in an created subcutaneous pouch, the implantable pulse generator (1) is connected to an extraocular muscle (4) by the electrode (3), and the implantable pulse generator (1) is for generating an pulse stimulating signal;
the electrode (3) comprises a lead (7) and a contact-pin connector (5) and a contact (6) that are provided at two ends of the lead, wherein the contact-pin connector (5) is connected to the implantable pulse generator (1), the lead (7) is provided in a created subcutaneous tunnel, and the contact (6) is connected to the extraocular muscle (4); and
the extracorporal remote controller (2) is connected to the implantable pulse generator (1) by a wireless signal, and is for setting the pulse stimulating signal of the implantable pulse generator (1);
wherein the implantable pulse generator (1) is for sensing a contraction state of an eye muscle by using the electrode (3), and according to the contraction state of the eye muscle generating an eye-muscle stimulating signal, and sending the eye-muscle stimulating signal to the extracorporal remote controller (2); and the implantable pulse generator (1) is further for receiving an eye-muscle stimulating signal of an opposite direction that is emitted by the extracorporal remote controller (2), and outputting the eye-muscle stimulating signal by using the electrode (3) to another eye muscle.

2. The implantable extraocular-muscle neuromuscular stimulator according to claim 1, wherein the implantable pulse generator (1) comprises a receiver, a transmitter, a wireless energy transfer module, a power management module, a digital processing module and an electrical-stimulation driver, and the wireless energy transfer module transmits energy to the power management module, the receiver and the transmitter; and the implantable pulse generator (1) wirelessly receives the signal by using the receiver, and transmits an output to the digital processing module, the signal is transmitted to the electrical-stimulation driver via a current generating circuit and a waveform time-sequence circuit, and the electrical-stimulation driver is connected to the electrode (3).

3. The implantable extraocular-muscle neuromuscular stimulator according to claim 1, wherein the extracorporal remote controller (2) comprises a receiver, a transmitter, a frequency synthesizer, a digital processing module, a data analysis module, a data storage module, a display and a processor, the processor is connected to the data analysis module, the data storage module, a controlling program and the display, and the frequency synthesizer is connected to the digital processing module, the receiver and the transmitter; the extracorporal remote controller (2) wirelessly receives the signal by using the receiver, and transmits the signal to the digital processing module, and the signal is transmitted to the processor via a digital interface circuit; and the processor transmits the signal to the data analysis module, and stores an analysis result to the data storage module, and the processor transmits the analysis result to the implantable pulse generator (1) via the digital interface circuit, the digital processing module and the transmitter.

4. The implantable extraocular-muscle neuromuscular stimulator according to claim 1, wherein the contact (6) on the electrode (3) is a plurality of contacts, and when the contacts (6) are connected to the extraocular muscle, at least one of the contacts (6) is connected to the extraocular muscle (4); and the contact (6) is connected to the extraocular muscle (4) by binding or insertion.

5. The implantable extraocular-muscle neuromuscular stimulator according to claim 1, wherein the setting of the pulse stimulating signal comprises: pulse amplitude setting, pulse width setting and pulse frequency setting.

6. The implantable extraocular-muscle neuromuscular stimulator according to claim 5, wherein the pulse amplitude setting comprises a voltage mode and a current mode, wherein when a pulse amplitude is set in the voltage mode, a voltage range is 0-10V, and an adjustment step length is 1V or 0.1V; and when a pulse amplitude is set in the current mode, a current range is 0-25 mA, and an adjustment step length is 1 mA or 0.1 mA.

7. The implantable extraocular-muscle neuromuscular stimulator according to claim 5, wherein a range of the pulse width setting is 30-450 ms, and an adjustment step length is 10 ms.

8. The implantable extraocular-muscle neuromuscular stimulator according to claim 5, wherein a range of the pulse frequency setting is 1-500 Hz.

9. A method for setting a parameter of the implantable extraocular-muscle neuromuscular stimulator according to claim 1, wherein the setting the pulse stimulating signal of the implantable pulse generator (1) by using the extracorporal remote controller (2) comprises: pulse amplitude setting, pulse width setting and pulse frequency setting.

10. The method for setting a parameter of the implantable extraocular-muscle neuromuscular stimulator according to claim 9, wherein the pulse amplitude setting comprises a voltage mode and a current mode, wherein when a pulse amplitude is set in the voltage mode, a voltage range is 0-10V, and an adjustment step length is 1V or 0.1V; and when a pulse amplitude is set in the current mode, a current range is 0-25 mA, and an adjustment step length is 1 mA or 0.1 mA;
a range of the pulse width setting is 30-450 ms, and an adjustment step length is 10 ms; and
a range of the pulse frequency setting is 1-500 Hz.

11. A surgical method for treating congenital idiopathic nystagmus by using the implantable extraocular-muscle neuromuscular stimulator of claim 1 comprising steps as follows:
S1, peeling to expose the extraocular muscle 4;
S2, binding or inserting the electrode 3 on a peeled extraocular muscle 4;
S3, adjusting the direction of the electrode 3, to avoid the pulling and stress of the electrode 3, and fixing a section of the electrode 3 on the sclera by stitching;
S4, creating a subcutaneous pouch behind an ear for the implantable pulse generator 1;
S5, introducing the electrode 3 via the orbit tunnel and the subcutaneous tunnel to the subcutaneous pouch where the implantable pulse generator 1 is placed;
S6, connecting the implantable pulse generator 1 and the electrode 3, by ensuring that the contact-pin connector 5 on the electrode 3 is inserted into the top cover of the implantable pulse generator 1, and screwing up a fastening screw on the top cover of the implantable pulse generator 1;
S7, performing system test to the implantable pulse generator 1 by using the extracorporal remote controller 2, to confirm that the system is functioning normally;
S8, encasing the implantable pulse generator 1 into the subcutaneous pouch, and Winding the redundant lead around the implantable pulse generator 1;
S9, stitching a stitching hole on the implantable pulse generator 1 and the subcutaneous tissue together, and fixing the implantable pulse generator 1 at the position of the subcutaneous pouch;
S10, performing system test to the implantable pulse generator 1 by using the extracorporal remote controller 2 again, to confirm that the system is functioning normally; and
S11, disinfecting, and stitching the surgical incision.
